(19) United States
(12) Patent Application Publication
(10) Pub. No.: US 2014/0067029 A1
(43) Pub. Date: Mar. 6, 2014

(54) RENAL NERVE MODULATION AND ABLATION CATHETER ELECTRODE DESIGN

(71) Applicant: BOSTON SCIENTIFIC SCIMED, INC., MAPLE GROVE, MN (US)

(72) Inventors: TRAVIS J. SCHAUER, DELANO, MN (US); GORDON J. KOCUR, LINO LAKES, MN (US)

(73) Assignee: BOSTON SCIENTIFIC SCIMED, INC., MAPLE GROVE, MN (US)

(21) Appl. No.: 14/012,767

(22) Filed: Aug. 28, 2013

Related U.S. Application Data
(60) Provisional application No. 61/694,087, filed on Aug. 28, 2012.

(51) Int. Cl. A61N 1/05

(52) U.S. Cl. CPC A61N 1/05
USPC 607/116

(57) ABSTRACT

An intravascular nerve modulation or tissue/ablation heating system comprising an elongate shaft having a proximal end region and a distal end region, a plurality of electrodes disposed adjacent the distal end region, wherein the plurality of electrodes are configured to operate in phase.

### CROSS-REFERENCE TO RELATED APPLICATIONS

This application claims priority under 35 U.S.C. §119 to U.S. Provisional Application Ser. No. 61/694,087, filed Aug. 28, 2012, the entirety of which is incorporated herein by reference.

### TECHNICAL FIELD

The present invention relates to methods and apparatuses for nerve modulation techniques such as ablation of nerve tissue or other destructive modulation technique through the walls of blood vessels and monitoring thereof.

### BACKGROUND

Certain treatments require the temporary or permanent interruption or modification of select nerve function. One example treatment is renal nerve ablation which is sometimes used to treat hypertension and other conditions related to hypertension and congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular renal nerves using a radio frequency (RF) electrode in an off-wall configuration or in a configuration in contact with the vessel wall. RF electrodes may ablate the perivascular nerves, but may also damage the vessel wall or other tissue in the area as well. Control of the ablation may effectively ablate the nerves while minimizing injury to the vessel wall. Sensing electrodes may allow the use of impedance measuring to monitor tissue changes. It is therefore desirable to provide for alternative systems and methods for intravascular nerve modulation.

### SUMMARY

The disclosure is directed to several alternative designs, materials and methods of manufacturing medical device structures and assemblies for performing and monitoring tissue changes.

Accordingly, one illustrative embodiment is a system for nerve modulation that includes a plurality of electrodes at a distal end region. The electrodes may be circumferentially arranged or may be arranged in a spiral or in another suitable location. The system includes one or more sources of power and is configuration such that the electrodes may supply energy in phase. In some embodiments, the energy to each of the electrodes may be separately deliverable such that the power to each of the electrodes may be separately varied. A separate conductor may extend between each of the electrodes and the power supply. Each of the conductors may be the same length to ensure the electrodes are in phase.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a distal end of an illustrative renal nerve modulation system.

FIG. 3 illustrates a distal end of an illustrative renal nerve modulation system.

FIG. 4 illustrates a distal end of an illustrative renal nerve modulation system.

FIG. 5 illustrates a distal end of an illustrative renal nerve modulation system in situ.

FIG. 6 illustrates the heating effect of a single electrode renal nerve modulation system in situ.

FIG. 7 illustrates the heating effect of a four-electrode wall-contacting renal nerve modulation system in situ where the electrodes are operating in phase.

FIG. 8 illustrates the heating effect of a four-electrode wall-sparing renal nerve modulation system in situ where the electrodes are operating in phase.

FIG. 9 illustrates the heating effect of a four-electrode helical renal nerve modulation system in situ where the electrodes are operating in phase.

FIG. 10 illustrates a distal end of an illustrative renal nerve modulation system.

FIG. 11 illustrates a distal end of an illustrative renal nerve modulation system.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed. As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired. For example, the devices and methods described herein may also be used for prostate ablation, tumor ablation, sympathetic nerve ablation, and/or other therapies requiring heating or ablation of target tissue. In some instances, it may be desirable to ablate perivascular renal nerves with deep target tissue heating. As energy passes from a modulation element to the desired treatment region the energy may heat both the tissue and the intervening fluid (e.g. blood) as it passes. As more energy is used, higher temperatures in the desired treatment region may be achieved thus resulting in a deeper lesion. Monitoring tissue properties may, for example, verify effective ablation, improve safety, and optimize treatment time. The term ablation is intended to refer to any tissue modulation process where the properties of the tissue may be altered.

In some instances, impedance monitoring may be used to detect changes in target tissues as ablation progresses. Sensing electrodes may be provided in addition to the modulation element. In some instances, the impedance may not be directly measured, but may be a function of the current distribution between the sensing electrodes. In general, the resistance of the surrounding tissue may decrease as the temperature of the tissue increases until a point where the tissue begins to denature or irreversibly change, for example, at approximately 50-60° C. Once the tissue has begun to denature the resistance of the tissue may increase. As the target tissue is ablated, the change in impedance may be analyzed to determine how much tissue has been ablated. The power level and duration of the ablation may be adjusted accordingly based on the impedance of the tissue. In some instances, overall circuit impedance may be monitored and modulation systems may utilize a standard power delivery level, but variation in local tissue impedance can cause unpredictable variation in the ablation effect on the target tissue and in local artery wall heating. It may be desirable to provide a simple way to determine local tissue impedance in order to control ablation using a split electrode.

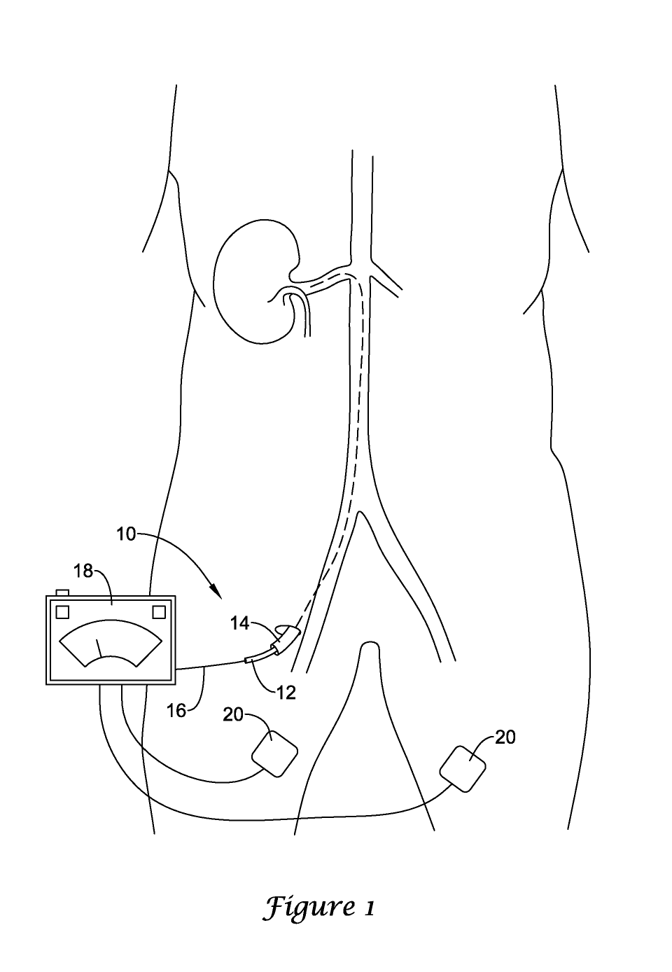

FIG. 1 is a schematic view of an illustrative renal nerve modulation system 10 in situ. System 10 includes a device 12 that includes one or more conductors 16 for providing power to one or more electrodes (not illustrated) disposed within the device 12. The system 10 may include other elements such as a delivery catheter 14. A proximal end of conductor(s) 16 may be connected to a control and power element 18, which supplies the necessary electrical energy to activate the one or more electrodes in the distal end region of the device 12. In some instances, return electrode patches 20 may be supplied on the patient's back or at another convenient location on the patient's body to complete the circuit. In other embodiments, the device 12 may include one or more pairs of bipolar electrodes. The control and power element 18 may include monitoring elements to monitor parameters such as power, temperature, voltage, pulse size and/or shape and other suitable parameters as well as suitable controls for performing the desired procedure. In some instances, the power element 18 may control a radio frequency (RF) electrode. The electrode may be configured to operate at a frequency of approximately 460 kHz. It is contemplated that any desired frequency in the RF range may be used, for example, from 450-500 kHz. Lower or higher frequencies may be used, such as 10 kHz or 1000 kHz, in some cases, although the desired heating depth, catheter size, or electrical effects can limit the choice of frequency. However, it is contemplated that different frequencies of energy outside the RF spectrum may be used as desired, for example, but not limited to, microwave.

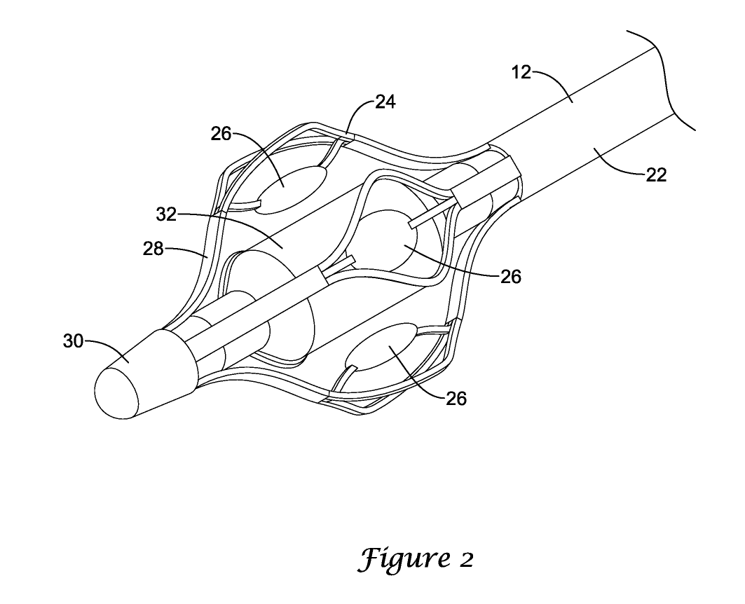

Embodiments pertain to the optimization of energy delivery through a multiple electrode renal nerve modulation system. Accordingly, many different renal nerve modulation systems may be suitable. For example, FIG. 2 illustrates the distal end of a wall-sparing device 12 that includes an ablation device 24 on the distal region of a catheter 22. The ablation device 24 includes a plurality of electrodes 26 (four are illustrated in this particular embodiment) on an expandable strut assembly 28. The expandable strut assembly may be biased to the expanded position as illustrated. The ablation device may include other features such as a partial occlusion spacer 32 and an atraumatic distal end 30.

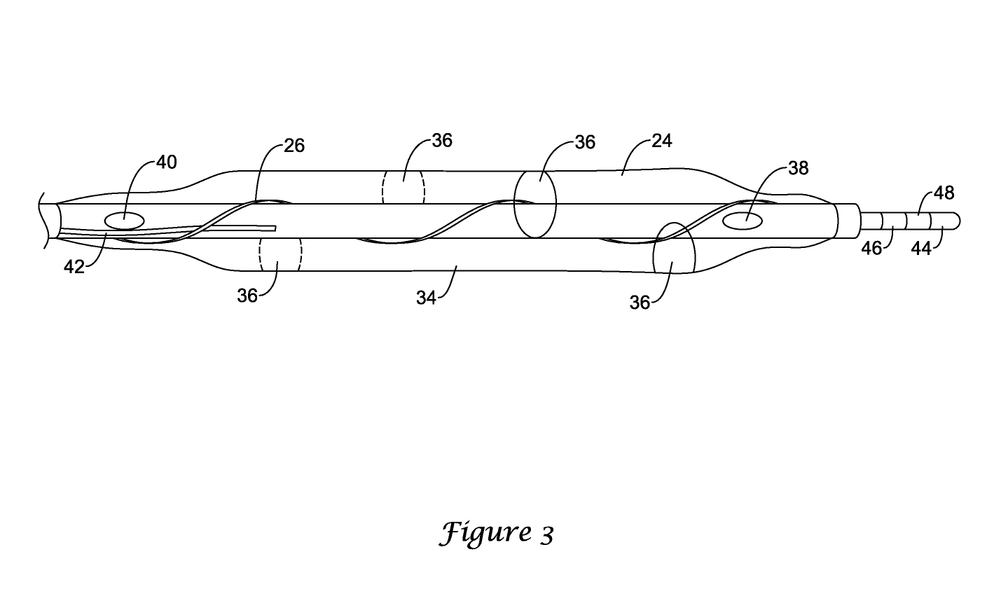

FIG. 3 illustrates the distal end of another embodiment that may be suitable. The embodiment of FIG. 3 is a virtual electrode embodiment, where the renal nerve ablation device 24 has an electrode 26 in an expandable balloon 34. The balloon 34 is generally made from a non-electrically conductive material except for windows 36 through which the energy is transmitted. The device may further include a fluid inlet lumen 38, a fluid outlet lumen 40, and one or more temperature sensors 42. A guidewire 44 may extend through a central lumen of the device. An ultrasonic transducer 48 may be disposed on the guidewire or at other suitable locations. (The ultrasonic transducer 48 will be discussed below.)

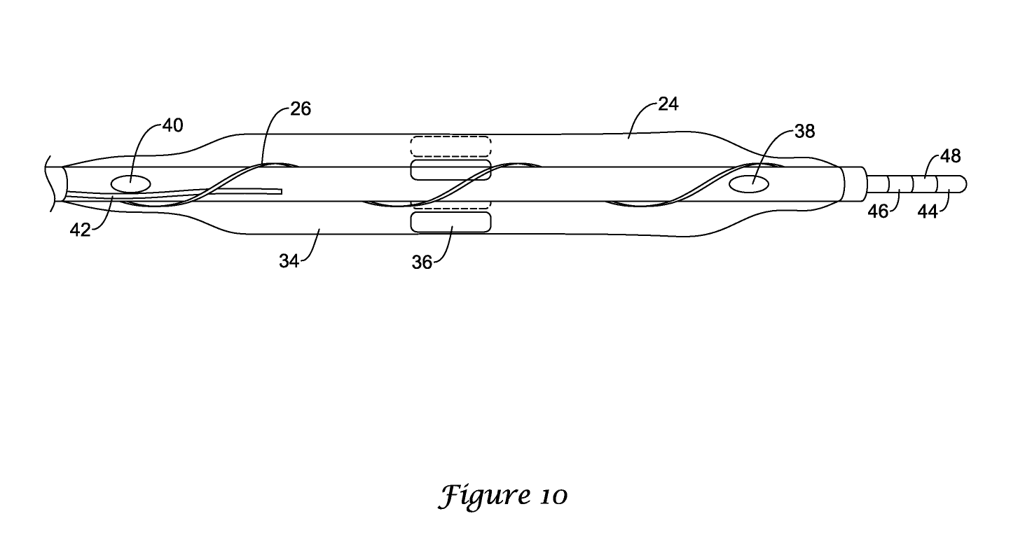

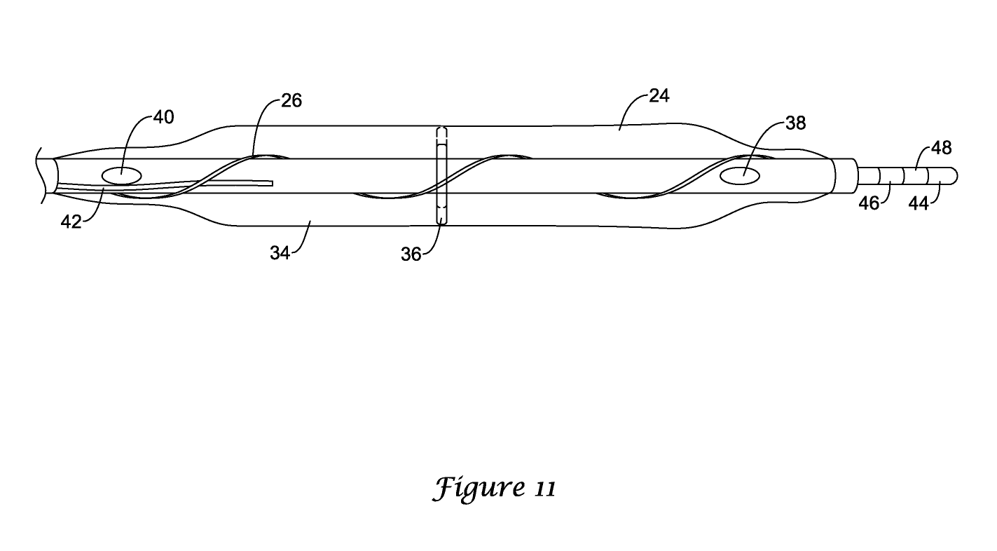

FIGS. 10 and 11 illustrate contemplated variations of the FIG. 3 embodiment where the windows 36 are arranged in a circumferential pattern. In FIG. 10, four windows 36 of about 2 mm by 4 mm are equally spaced about a balloon of approximately 5 mm in diameter. In FIG. 11, two windows of about 1 mm by 4 mm are equally spaced about a balloon of about 5 mm diameter. It will be observed that the major dimensions of the windows extend axially in the FIG. 10 embodiment and circumferentially in the FIG. 11 embodiment.

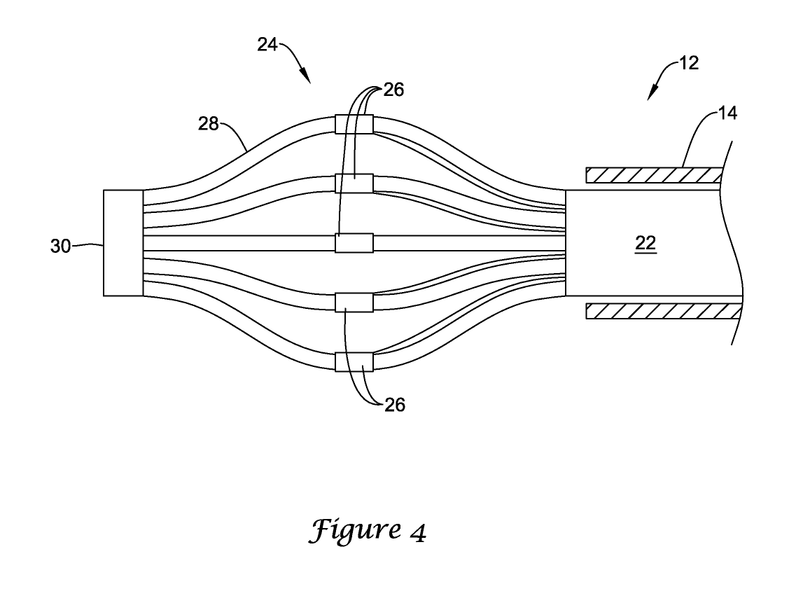

FIG. 4 illustrates the distal end of another suitable embodiment. A plurality of electrodes 26 may be disposed on a strut assembly 28, which is captured between the distal end 30 of the system and a catheter 22. The electrodes are circumferentially arranged such that they contact the vessel wall when the distal portion of the device is expanded in situ.

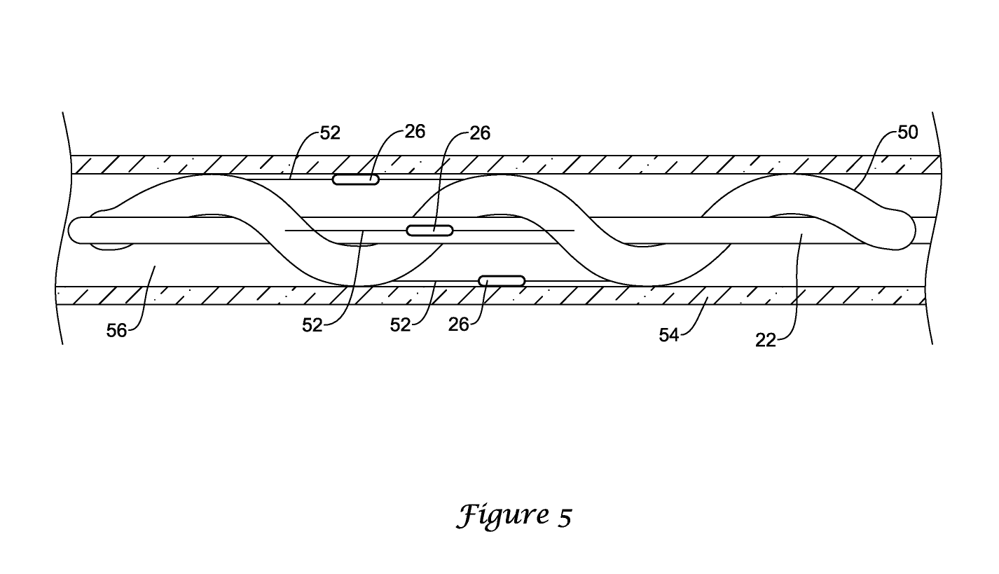

FIG. 5 illustrates the distal end of another suitable embodiment. In this embodiment, an expandable helical balloon 50 is disposed about a catheter 22. Electrodes 26 are disposed on struts 52 between loops of the helical balloon and may be arranged in a helical pattern. There may be, for example, four electrodes 26 spaced at 90 degrees from adjacent electrodes. The electrodes 26 may be arranged so that they contact the vessel wall 54 or are spaced from the vessel wall in the vessel lumen 56.

In at least some of these embodiments, it is contemplated that power may be supplied to the electrodes such that the power radiates from the electrodes in phase. This permits the electrical fields from the separate electrodes to advantageously interact to provide an optimized heating pattern. In some systems, for example, total power needed is reduced and the tissue is exposed to lower power and experiences lower (but still effective) temperatures. In some systems, this may require separate conductors, with a separate conductor extending from the power supply to each of the electrodes. In some systems, the conductors may each be the same length. In other systems, a separate power source is provided for each electrode. This separate power source may be a separate power generator for each electrode or may be a common power generator with an intervening controller that provides for separate power to each of the electrodes. In such systems the power to each of the electrodes may be varied.

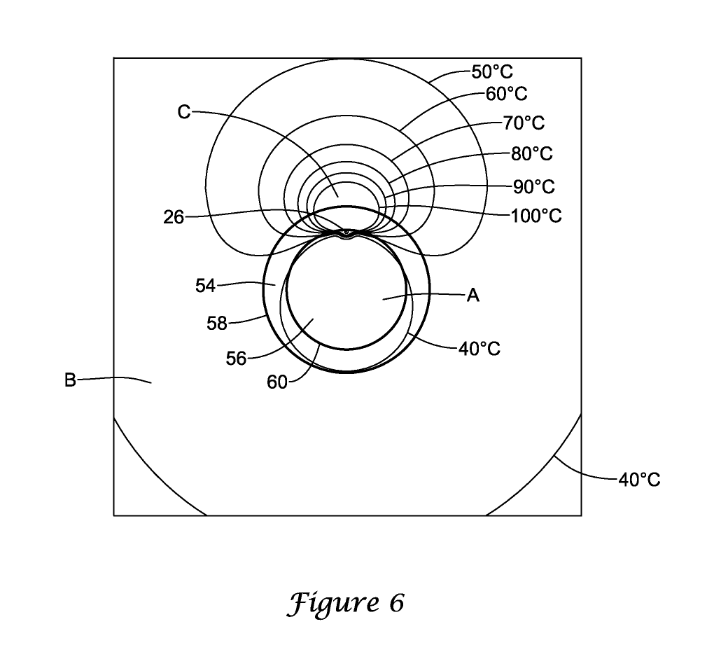

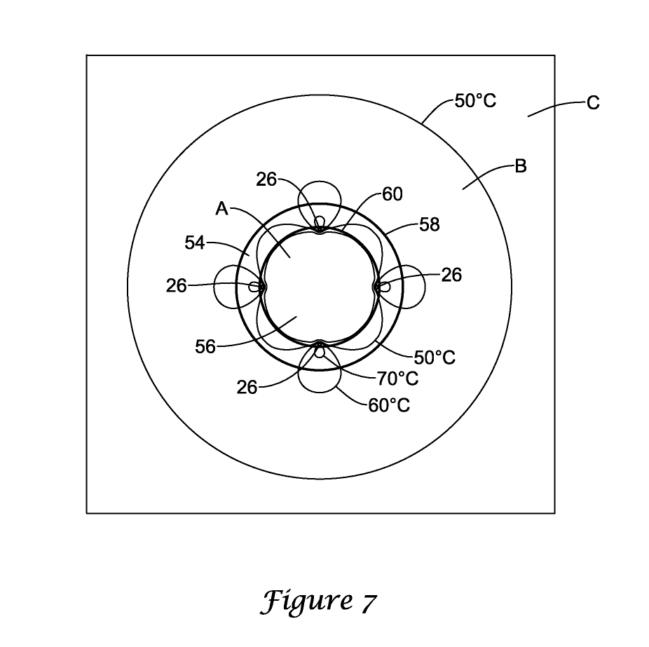

A comparison of FIGS. 6 and 7 may illustrate the advantages of the present invention. FIG. 6 illustrates a single electrode system, with the electrode 26 against the inner wall 60 of a blood vessel 54. The lumen 56 and outer wall 58 are also illustrated. The isotherms illustrate the heating pattern created by supplying RF energy through this single electrode. It can be seen that a large portion, indicated at “A” and including most of the lumen 56 is below 40° C., a second substantial portion, indicated at “B” is between 40° C. and 50° C., and a portion near the electrode, indicated at “C” is about 100° C. In contrast, FIG. 7 illustrates a four-electrode configuration, where the same amount of total power is provided through four equally spaced electrodes 26 that also are against the vessel wall 54. The power through the four electrodes 26 is in phase. In FIG. 7, nearly the whole of the lumen 56 (the region indicated at “A”) is below 40° C., a large uniform portion “B” is between 50° C. and 60° C. and the further tissue “C” is at 50° C. or below. Further, the maximum temperature reached is substantially less, about 87° C. A much more uniform temperature pattern is observed in FIG. 7 than in FIG. 6.

Suitable electrode arrays may be designed with the following considerations. An electrode array length of about or less than 20 mm will be long enough to treat most human renal arteries in one application or in multiple applications. Array length may be adjusted to vary maximum treatment depth. Lengthening the array may increase the maximum treatment depth and shortening the array may decrease the maximum treatment depth. Electrode array diameters of between 4 mm and 8 mm will be suitable to treat most human renal arteries. Multiple array configurations, having different array sizes and electrode sizes, may be desirable to treat the range of vessel diameters and ensure electrical field interactions. Electrode sizes or diameters of between about 0.05 mm and 1.4 mm may be suitable for 6F compatible arrays. A particular power should be selected for an electrode of a given size. In one suitable configuration, the power is selected such that, at a tissue depth of 2 mm, a temperature of between about 50° C. and 90° C. is produced, and at a tissue depth of greater than 4 mm, a temperature of no greater than 65° C. is produced. In some arrays a suitable spacing pattern between electrodes may be produced by limiting axial spacing between adjacent electrodes to less than about 4 mm and circumferential spacing to less than about 10 electrode diameters.

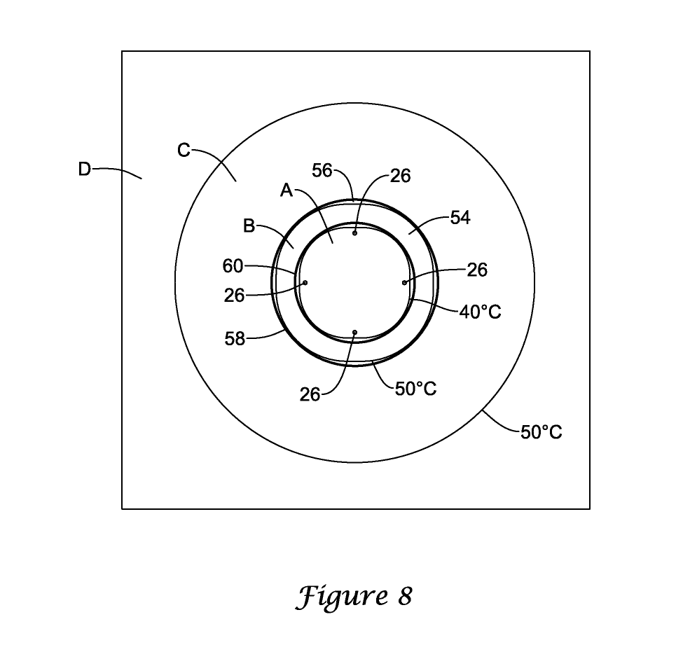

FIG. 8 illustrates a configuration where the four electrodes are spaced from the vessel wall. The power provided through the electrodes is same as in the FIG. 6 and FIG. 7 examples and is in phase. A more uniform temperature pattern is observed, with the greater portion “B” of the wall of the vessel 54 between 40° C. and 50° C. and a uniform area “C” reaching a maximum temperature of 53° C. This temperature is sufficient for nerve treatment, while also avoiding traumatic tissue damage such as collagen denaturation, carbonization or water vaporization.

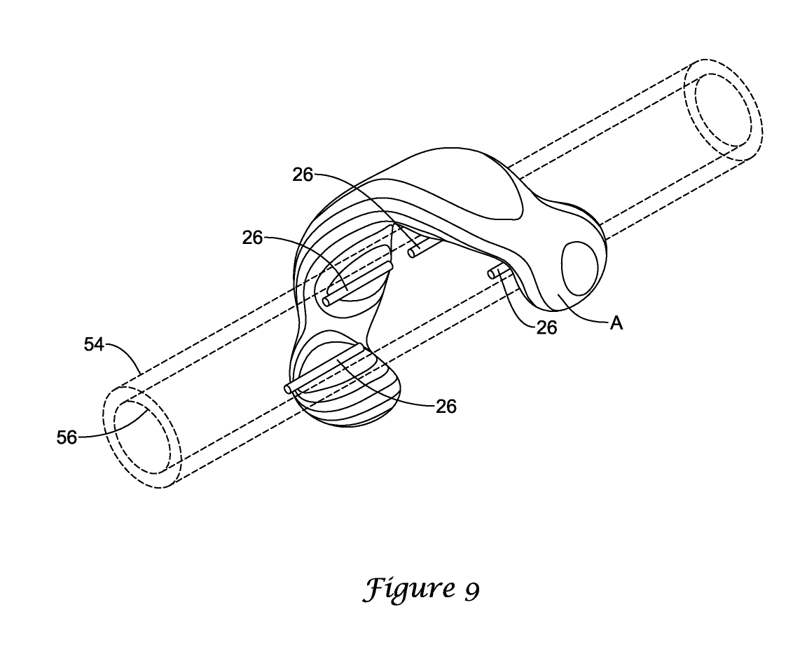

FIG. 9 illustrates that uniformity of temperature may also be achieved in a helical configuration. In FIG. 9, the vessel wall 54 is shown in broken lines, and only the electrodes 26 are illustrated of the system. It can be appreciated that the electrodes 26 may be part of a system such as that illustrated in FIG. 5. The region “A” illustrates where temperatures greater than 50° C. are achieved by in-phase RF power from the electrodes 26. (The solid lines of the region “A” are not isotherms; rather they indicate the distance from the vessel wall 54.)

It will be appreciated that the effective zones may be varied by varying the power to the electrodes. For example, in the FIG. 9 illustration, the power to the end electrodes may be varied to produce a more uniform effective zone “A”. A larger hot zone may be created by increasing the power to each of the electrodes. In some embodiments, the power supplied to the electrodes may be linked to temperatures sensed at each of the electrodes. The power supplied to the electrodes may be reduced should a preselected temperature be reached at one or more of the electrodes.

The temperature profile may be varied through other means as well. Returning to FIG. 3, which illustrates an ultrasonic transducer 48 disposed on a guidewire 44, which may be used to increase the denervation effect at a particular location without increasing the RF energy supplied. Ultrasonic energy and electromagnetic energy do not interfere with each other. Thus, if there is reason to provide additional denervation effect, an ultrasonic transducer may be suitable. The ultrasonic transducer 48 is preferably directional and may be focused to the desired depth. The ultrasonic transducer may be mounted on a separate element such as a guidewire 44 so that it may be moved and/or rotated to a desired location for treatment. Alternatively, the ultrasonic transducer may be fixed to the distal region of the device and the device may be relocated so that the ultrasonic transducer may be operated in an optional separate step once the electromagnetic portion of the procedure is done.

In use, any of the systems described herein may be advanced through the vasculature in any manner known in the art. For example, system 10 may include a guidewire lumen to allow the system 10 to be advanced over a previously located guidewire. In some embodiments, the modulation system 10 may be advanced, or partially advanced, within a guide sheath such as the guide catheter 14 shown in FIG. 1. Once the distal end region of the device 12 is placed adjacent to a desired treatment area, the guide catheter may be at least partially withdrawn to expose the distal end region. A deflection member may be actuated to position the distal end region near a treatment site. The electrode may be activated to provide RF energy. Nerve tissue may be heated by the RF energy and denatured or ablated. Once a particular spot has been treated, the distal end region of the catheter may be moved to treat a second location. For example, the distal end region may be rotated and/or deflected to treat a second location on the same circumferential region of the vessel wall or may be rotated and withdrawn proximally to treat a second location on a different circumferential region of the vessel wall spaced longitudinally and circumferentially from the first treated location. This procedure may be repeated until a desired number of locations have been treated. In some instances, it will be desirable to treat a vessel wall such that the complete circumference of a vessel wall is treated. This circumferential coverage may be provided by treating regions that are spaced longitudinally from each other and are at different circumferential locations or may be provided by treating a complete circumferential ring of the vessel wall.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. An intravascular nerve modulation system, comprising:
an elongate shaft having a proximal end region and a distal end region;
a first electrode disposed adjacent the distal end region;
a second electrode disposed adjacent to the first electrode; and
wherein the first electrode and the second electrode are configured to operate in phase.

2. The system of claim 1, further comprising a third electrode, wherein the first, second, and third electrodes are configured to operate in phase.

3. The system of claim 2, further comprising a fourth electrode, wherein the first, second, third, and fourth electrodes are configured to operate in phase.

4. The system of claim 1, wherein the first electrode and the second electrode are positioned the same longitudinal distance from the proximal end region and at different radial locations.

5. The system of claim 1, wherein the first electrode and the second electrode are positioned at different longitudinal locations.

6. The system of claim 1, wherein the distal end region includes a plurality of electrodes that are helically arranged about the shaft.

7. The system of claim 1, wherein the shaft includes a plurality of struts and wherein the first electrode, the second electrode, or both are disposed along the struts.

8. The system of claim 1, wherein the shaft includes an expandable balloon and wherein the first electrode, the second electrode, or both are disposed along the balloon.

9. The system of claim 1, wherein the shaft includes a helical balloon.

10. The system of claim 1, further comprising an ultrasonic transducer disposed adjacent to the shaft.

11. The system of claim 10, wherein the ultrasonic transducer comprises a focusable array.

12. The system of claim 10, wherein the ultrasonic transducer is slidable proximally and distally in the distal end region.

13. The system of claim 10, wherein the ultrasonic transducer is rotatable.

14. The system of claim 10, wherein the ultrasonic transducer is disposed on a guide wire.

15. The system of claim 1, further comprising a temperature sensor disposed adjacent to the first electrode.

16. The system of claim 1, wherein the power supplied to the first electrode and to the second electrode can be varied separately.

17. An intravascular nerve modulation system, comprising:
an elongate shaft having a proximal end region and a distal end region;
a helical balloon coupled to the distal end region, the helical balloon including a plurality of loops;
a plurality of struts extending between the loops of the helical balloon;
wherein the plurality of struts include a first strut and a second strut;
a first electrode disposed along the first strut;
a second electrode disposed along the second strut; and
wherein the first electrode and the second electrode are configured to operate in phase.

18. The system of claim 17, wherein the first electrode, the second electrode, or both are designed to contact a vessel wall when the balloon is expanded.

19. The system of claim 17, wherein the first electrode, the second electrode, or both are designed to be positioned radially inward from a vessel wall when the balloon is expanded.

20. A method of tissue modulation, the method comprising:
providing an intravascular nerve modulation system, the system comprising:
an elongate shaft having a proximal end region and a distal end region,
a first electrode disposed adjacent the distal end region,
a second electrode disposed adjacent to the first electrode, and
wherein the first electrode and the second electrode are configured to operate in phase;
advancing the distal end region of the shaft through a blood vessel to a position adjacent to a region of interest; and
activating the first electrode and the second electrode in phase.
